(19) United States
(12) Patent Application Publication
(10) Pub. No.: US 2008/0234504 A1
(43) Pub. Date: Sep. 25, 2008

(54) Processes for preparation of 9-haloacetamidominocyclines

(76) Inventors: Evgeny Tsiperman, Be'er Sheva (IL); Sofia Gorohovsky, Be'er Sheva (IL); Slavik Yurkovski, Kiryat-Gat 8 (IL); Sergei Fine, Qiriat-Arbaa (IL); Tamas Koltai, Netanya (IL); Judith Aronhime, Rehovot (IL)

Correspondence Address: KENYON & KENYON LLP, ONE BROADWAY, NEW YORK, NY, 10004, US

(21) Appl. No.: 11/998,825

(22) Filed: Nov. 30, 2007

Related U.S. Application Data
(60) Provisional application No. 60/872,033, filed on Nov. 30, 2006.

(51) Int. Cl. C07C 237/26

(52) U.S. Cl. 552/205

(57) ABSTRACT

The present invention provides substantially pure intermediates, 9-haloacetomidominocyclines, and process of preparing them that are useful for the preparation of glycylcyclines, specifically Tigecycline.

### CROSS-REFERENCE TO RELATED APPLICATIONS

This application claims the benefit of U.S. Provisional Application No. 60/872,033, filed Nov. 30, 2006. The contents of which are incorporated herein by reference.

### FIELD OF THE INVENTION

The invention is directed to an improved processes for preparing 9-haloacetamidominocyclines, such as 9-chloroacetamidominocyclines and 9-bromoacetamidominocyclines which are useful as intermediates for preparing glycylcyclines such as Tigecycline.

### BACKGROUND

Tigecycline (CAS 220620-09-7), (4S,4aS,5aR,12aS)-9-(2-(tert-butylamino)acetamido)-4,7-bis(dimethylamino)-1,4,4a,5,5a,6,11,12a-octahydro-3,10,12,12a-tetrahydroxy-1,11-dioxo-2-naphthacenecarboxamide, is the first drug of a new generation of tetracycline antibiotics called glycylcyclines. Tigecycline has a wider range of bioactivity than the parent tetracycline and its analogues discovered so far, such that it may be administrated less frequently and/or in lower dose.

Tigecycline has been introduced and marketed by Wyeth under the brandname TYGACIL® and is especially indicated against acute lethal infections caused by Gram-negative bacteria. TYGACIL® is marketed as leophilized powder or cake for intravenous injection.

Tigecycline has the following structure:

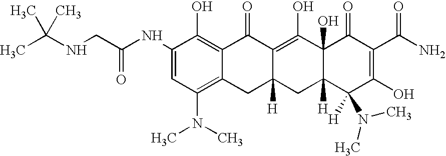

### Tigecycline: C₂₉H₃₉N₅O₈

### MW: 585.65 g/mol

This molecule was disclosed in U.S. Pat. No. 5,494,903, while processes for its preparation are described in U.S. Pat. No. 5,675,030.

As referred to in U.S. Pat. No. 5,675,030, the tetracycline molecule presents special challenges to the synethetic organic chemist. The molecule can be readily oxidized at the C-11 and C-12a positions. In addition, when there is a 7-distributed amino group, the D ring is an aminophenol which is prone to oxidation. The molecules can epimerize at the C-4 position of the D ring with the resultant decrease in bacterial activity. Epimerization at the C-4 position can occur at any stage utilized to prepare tigecycline. Factors which increase epimerization apparently include mildly acidic conditions, temperature above 25° C. and the presence of moisture in the reaction. Ultimately, the C-4 epimer can vary from 1-50%.

Important in the preparation of Tigecycline are intermediates including 9-chloro and 9-bromoacetamidominocycline. U.S. Pat. No. 5,494,903, examples 25, 98, 99 and 101 describe preparation of both 9-chloro and 9-bromoacetamidominocycline intermediates in the form of free base or acid addition salt, where the acid addition is characterized by mass-spectroscopy.

Tetracyclines, in general, show relatively little tendency to extract at any pH into common water immiscible organic solvents such as diethyl ether and chloroform and alike. [L. A. Mitscher, The chemistry of the Tetracycline Antibiotic. (1978) Marcel Dekker Inc.]. However, in U.S. Pat. No. 5,675,030, Example 7, the 9-chloroacetamidominocycline is produced by a reaction which is then quenched by a basic aqueous solution, extracted by methylene chloride, and precipitated using a haptane:iso-propanol mixture. The resultant is described as “an impure material contaminated with a mixture of esters,” which requires hydrolysis in the next stage, and apparently necessitates the use of a resin to purify the tigecycline prepared, as described in Example 8.

Because 9-chloro and 9-bromoacetamidominocycline are amphoteric, i.e., behaving both as an acid or base and possessing functional groups that can chelate readily, many of the conventional purification techniques for organic compounds, such as chromatography on silica gel or preparative HPLC, cannot be applied to their purification.

Thus, there is a need in the art for improved methods of obtaining substantially pure 9-chloro and 9-bromoacetamidominocycline.

### SUMMARY OF THE INVENTION

This invention provides a simple and feasible method of preparation of Tigecycline of high purity in improved yield. The said method requires using a pure intermediate that can be prepared according to another aspect of this invention.

The present invention encompasses solid and/or isolated (4S,4aS,5aR,12aS)-9-haloacetamido-4,7-bis(dimethylamino)-1,4,4a,5,5a,6,11,12a-octahydro-3,10,12,12-tetrahydroxy-1,11-dioxo-2-naphthacenecarboxamide, referred to herein as 9-haloacetamidominocycline, including 9-chloracetamidominocycline and 9-bromoacetamidominocycline, in the form of free acid or amine addition salt.

The present invention further encompasses substantially pure 9-haloacetamidominocycline, including 9-chloracetamidominocycline and 9-bromoacetamidominocycline, both in the form of free acid and amine addition salt.

In another aspect of the present invention, a process is presented for preparing solid, isolated and substantially pure 9-haloacetamidominocycline including 9-chloracetamidominocycline and 9-bromoacetamidominocycline, both in the form of free acid, and amine addition salt. This process comprises: providing a solution comprising 9-haloacetomidominocycline, preferably 9-chloroacetamidominocycline or 9-bromoacetominocycline; adjusting or maintaining the pH between about 4 to about 7, preferably about 5 to about 6, more preferably about 5.0 to about 5.6; using a water immiscible organic solvent to extract substantially pure 9-haloacetmidominocycline and optionally recovering solid and/or isolated substantially pure 9-haloacetamidominocycline.

In another embodiment, this invention encompasses a process for preparing 9-haloacetamidominocycline, preferably 9-chloroacetamidominocycline or 9-bromoacetamidominocycline in free acid form comprising: providing an organic solution of 9-haloacetamidominocycline, preferably 9-chloroacetamidominocycline or 9-bromoacetamidominocycline; reducing the volume of the solution; admixing at least 3 equivalents of a C₅-C₈ saturated hydrocarbon, preferably n-hexane, or cyclohexane to obtain a precipitate; and recovering the precipitated 9-halooacetominocycline, preferably 9-chloroacetamidominocycline or 9-bromoacetamidominocycline in free acid form. Recovery may be by any means known in the art such as by filtering, followed by drying over night under vacuum, such as at a temperature of about 40° C.

In another embodiment, this invention encompasses a process for preparing 9-haloacetominocycline, preferably 9-chloroacetominacycline or 9-bromoacetamidominocycline in salt or adduct form comprising: providing an organic solution of substantially pure 9-chloroacetamidominocycline and mixing about 1 to about 20 molar equivalents of an amine including, but not limited to t-butylamine, triethylamine, isopropylamine, hydrochloric acid, hydrobromic acid and trifluoroacetic acid; and recovering substantially pure 9-haloacetamidominocycline in salt or adduct form.

The substantially pure 9-haloacetamidominocycline of the present invention can be further converted into glycylcyclines, such as and Tigecycline. manufacture of a pharmaceutical composition.

### BRIEF DESCRIPTION OF THE FIGURES

FIG. 1 illustrates a powder X-ray diffraction pattern for the isolated t-butylammonium salt of 9-chloroacetamidominocycline (as prepared by example 3).

FIG. 2 illustrates a powder X-ray diffraction pattern for the isolated 9-chloroacetamidominocycline as a free acid (as prepared by example 4).

### DETAILED DESCRIPTION OF THE INVENTION

As used herein, unless specified otherwise, “substantially pure” is meant to be at least 90% pure by area as determined by HPLC. The substantially pure 9-haloacetamidominocycline of the present invention is preferably more than 95% pure by area and more preferably more than 97% pure by area, and most preferably more than 99% pure by area as determined by HPLC. In addition, the substantially pure 9-haloacetamidominocycline of the present invention may be substantially free of the corresponding C-4 epimer,

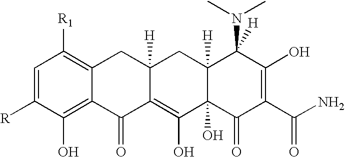

### C-4 Epimer

wherein R₁ is a dialkyl amino and R is a 2-(t-butylamino)-acetamido group. As used herein the term “substantially free of the corresponding epimer” is meant to refer to having not more than 10% of the C-4 epimer. The substantially pure 9-haloacetamidominocycline, substantially free of the corresponding C-4 epimer, preferably has not more than 5% of the C-4 epimer, more preferably not more than 3% of the C-4 epimer, and most preferably not more than 1% of the C-4 epimer.

The present invention encompasses substantially pure 9-haloacetmidominocycline, including 9-chloracetamidominocycline and 9-bromoacetamidominocycline, both in the form of free acid and amine addition salt. The present invention further encompasses solid and/or isolated (4S,4aS,5aR,12aS)-9-haloacetamido-4,7-bis(dimethylmino)-1,4,4a,5,5a,6,11,12a-octahydro-3,10,12,12a-tetrahydroxy-1,11-dioxo-2-naphthacenecarboxamide, referred to herein as 9-haloacetamidominocycline, including 9-chloracetamidominocycline and 9-bromoacetamidominocycline, both in the form of free acid and amine addition salt. The 9-haloacetamidominocycline may be represented by the following formula

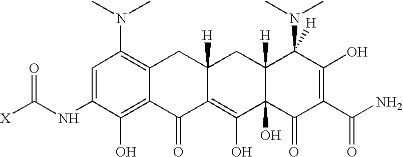

wherein X is a halogen.

In another aspect of the present invention, a process is presented for preparing substantially pure 9-haloacetamidominocycline as well as solid and/or isolated substantially pure 9-haloacetamidominocycline, both in the form of free acid, and amine addition salt. This process comprises: providing a solution comprising 9-haloacetomidominocycline, preferably 9-chloroacetomidominocycline or 9-bromoacetomidominocycline, in water; adjusting or maintaining the pH between about 4 to about 7, preferably about 5 to about 6, more preferably about 5.0 to about 5.6; and extracting substantially pure 9-haloacetamidominocycline, preferably 9-chloroacetamidominocycline or 9-bromoacetamidominocycline using a water immiscible organic solvent; and optionally recovering solid and/or isolated 9-haloacetamidominocycline in free acid form or further converting to amine addition salt or adduct form. The solution comprising 9-haloacetamidominocycline in water preferably further comprises a water miscible organic solvent, preferably a straight or cyclic C₃₋₇ amide organic solvent, more preferably the organic solvent is selected from the group consisting of DMI(1,3-dimethylimidazolidin-2-one), DMA (Dimethylacetamide), DMF (Dimethylformamide), NMP(N-methylpyrrolidone) and DMPU (N,N′-Dimethylpropyleneurea)

The solution comprising 9-chloroacetomidominocycline may be obtained as a result of a synthetic reaction. Alternatively, the solution comprising 9-chloroacetamidominocycline may be obtained by mixing an insufficiently pure solid 9-chloroacetmidominocycline with a solvent, wherein the solid 9-chloroacetmidominocycline has a purity less than desired for its intended purpose. A solid 9-chloroacetamidominocycline having a purity of less than 98% as measured by HPLC area percent may be in certain situations be considered insufficiently pure for its intended purpose. In one example, the mixture comprising 9-chloroacetamidominocycline may be prepared by reacting an acylating agent such as chloroacetic anhydride or chloroacetyl chloride, with 9-aminominocycline in an amide such as DMF, at a low temperature of less than about 10° C., more preferably about 0-5° C., even more preferably about 0-2° C. and pouring the mixture into water, preferably ice cold water. This process is similar to the process described in for example Example 3 of U.S. Pat. No. 5,675,030, which reference is incorporated herein in its entirety by reference, which example prepares [4S-(4-alpha,12aalpha)]-9-[(chloroacetyl)amino-4-(dimethylamino)-1,4,4a,5,5a,6,11,12a-octahydro-3,10,12,12a-tetrahydroxy-1,11-dioxo-2-naphthacenecarboxamide, a compound different than 9-chloroacetamidominocycline. However, in contrast to the '030 patent where the 9-chloroacetamidominocycline in a basic pH is obtained and then precipitated using a heptane: isopropanol mixture, the reaction mixture in the present invention is adjusted to an acidic pH and the 9-chloroacetamidominocycline is extracted using a water immiscible organic solvent. The same process to obtain a solution comprising 9-chloroacetamidominocycline may be employed to obtain any other 9-haloacetamidominocycline wherein the choro substituent is a different halogen.

Preferably, any inorganic or organic base or a basic aqueous solution can be used in the present invention to obtain the desired pH, while inorganic bases and their solutions are preferable. In one example, an ammonium hydroxide solution is used.

Water immiscible organic solvents may be selected from the group consisting of: a linear or branch-chain C₂₋₈ ether, linear or branch-chain C₃₋₆ ketones, linear or branch-chain C₅₋₁₂ esters, halogenated hydrocarbons and mixtures thereof. Preferably, the water immiscible organic solvents are selected from the group consisting of iso-butyl acetate, methyl iso-butyl ketone, methyl t-butyl ether, dichloromethane and mixtures thereof. Most preferably, dichloromethane is used.

Extracting 9-haloacetamidominocycline using a water immiscible organic solvent may be performed a number of times to obtain the desired yield and purity.

Recovering substantially pure 9-haloacetamidominocycline may include exposure to a drying agent such as sodium sulfate or magnesium sulfate prior to isolation of the 9-haloacylated product. For recovery, when the 9-haloacetomidominocycline is desired in the free acid form, the free acid is precipitated out. In one example, the recovery process of precipitating the 9-haloacetamidominocycline comprises: combining the solution containing the 9-haloacetamidominocycline with an antisolvent, preferably the antisolvent is a C₅-C₈ saturated hydrocarbon, more preferably n-hexane or cyclohexane. Preferably in the precipitation process of the present invention, the first solvent is replaced, for example by reducing the volume of the solution, and admixing at least a 3 fold amount in volume, with respect to the amount of the first solvent, of an antisolvent, preferably a C₅-C₈ saturated hydrocarbon, more preferably n-hexane or cyclohexane, to obtain a precipitate. The precipitate can then be filtered and dried over night under vacuum, such as at a temperature of about 40° C.

When the 9-haloacetomidominocycline is desired in amine addition salt or adduct form, the process may further comprise: admixing about 1 to about 20, preferably about 1 to about 10, more preferably about 2 to about 5 molar equivalents of an amine including, but not limited to, t-butylamine, triethylamine, isopropylamine, hydrochloric acid, hydrobromic acid and trifluoroacetic acid; and recovering substantially pure 9-haloacetamidomidominocycline in salt or adduct form.

The substantially pure 9-chloracetamidominocycline of the present invention can be further converted into glycylcyclines, in general, and Tigecycline, specifically, by any means known in the art, such as for example described in Example 8 of U.S. Pat. No. 5,675,030, which reference is incorporated herein in its entirety by reference. The Tigecycline prepared from the substantially pure intermediate can be effectively isolated from the reaction mixture without using resins and carrying out numerous extractions at different pH values as described in the prior art process. Additionally, this invention is likely to afford the target material in a higher yield, simpler work-up and reduces the production cost.

The 9-haloacetamidominocycline, preferably 9-chloroacetamidominocycline in the form of free acid or amine addition salt prepared according to any procedure of this invention can be further reacted to obtain Tigecycline, by any method known in the art, preferably as described for example in Example 8 of U.S. Pat. No. 5,675,030. The Tigecycline obtained is preferably substantially pure Tigecycline. This Tigecycline may have a reduced amount of residual solvents and/or related impurities.

### Instrumentation

### HPLC Method for Determination of Chromatographic Purity of CMI:

| | |
|---|---|
| HPLC Column: | YMC Basic, 3μ, 150 × 3.0 mm |
| Column temp: | 25° C. |
| Mobile Phase: | (A) 0.05% v/v Heptaflurobutyric acid, 0.01M |
| | NH₄Ac adjusted to pH 3.3 with Acetic Acid; |
| | (B) Acetonitrile |
| Gradient: | (0 min; 5% B)→ (30 min; 35% B)→(40 min; 70% B) |
| Flow: | 0.7 ml/min |
| Injection Volume: | 10 μL |
| Detector: | UV 248 nm |

### EXAMPLES

### Example 1

### Preparation of 9-Chloroacetamidominocycline Solution in DCM Using Chloroacatylchloride

9-aminominocycline was dissolved in DMF and the mixture cooled at 0-5° C. 2.5 eq. of chloroacetyl chloride were added to the mixture, which was then stirred for an hour while allowed to reach the ambient temperature. The reaction mixture was then poured into ice-cold water and the resulted solution was adjusted at pH ˜5.3 and extracted several times with dichloromethane. The combined organic extracts were washed with water, dried over sodium sulfate and filtered to afford a solution of pure 9-chloroacetamidominocycline. (Purity: >99% by area; Yield=90-95%).

### Example 2

### Preparation of 9-Chloroacetamidominocycline Solution in DCM Using Chloroacetic Anhydride

Cold DMF was mixed with the required amount of H₂SO₄ 98% and after about 10 min. 9-aminominocycline was added to the mixture. 2 eq. of chloroacetic anhydride were then added to the resulted suspension that was further stirred for an hour. Upon completion of the reaction the mixture was poured into ice-cold water and the resulted solution was adjusted at pH ˜5.3 and extracted several times with dichloromethane. The combined organic extracts were washed with water, dried over sodium sulfite and filtered to afford a solution of pure 9-chloroacetamidominocycline. (Purity: >99% by area; Yield 80-95%).

### Example 3

### Isolation of t-Butylammonium Salt of 9-Chloroacetamidominocycline

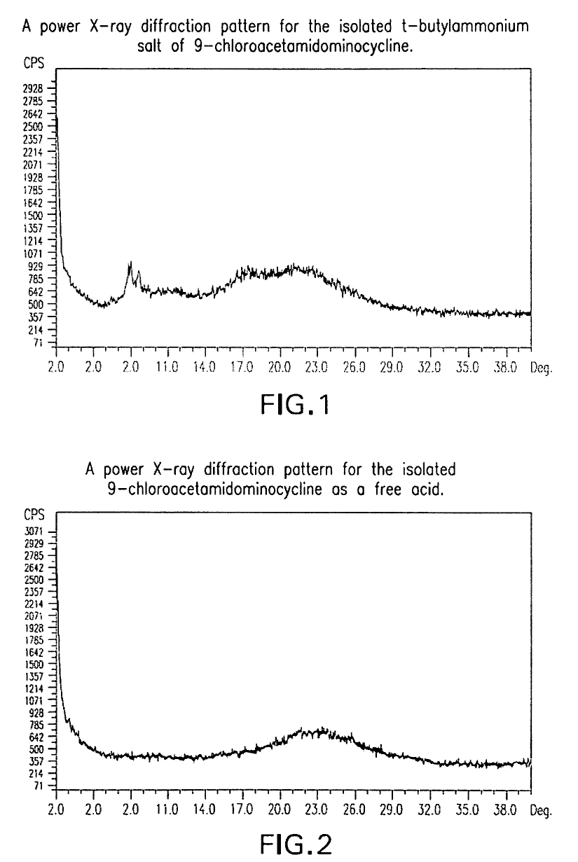

2 eq. of t-butylamine (based on the starting 9-aminominocycline) was added to the organic solution from Example 1 or 2. Precipitation started in a few minutes and the suspension was stirred for an hour. The solid was collected by vacuum filtration and dried under vacuum at 40° C. overnight. t-butylamine 9-chloroacetamidominocycline adduct thus obtained was characterized by chromatographic purity of >99% and the PXRD pattern of FIG. 1.

Samples of 9-chloroacetamidominocycline t-butyl amine adduct were analyzed by X-Ray Powder diffraction and found to contain an Amorphous Form with two peaks at 8.0, 8.7±0.2 degrees two theta.

### Example 4

### Isolation of 9-Chloroacetamidominominocycline as Free Acid

The organic solution of Example 1 or 2 was concentrated to a smaller volume and treated with at least trice mount of n-heptane to initiate precipitation. After stirring the suspension for an hour it was filtered and the solid dried overnight at 40° C. under vacuum to afford the desired product having high chromatographic purity and the PXRD pattern of FIG. 2:

A sample of 9-chloroacetamidominocycline free acid was analyzed by X-Ray Powder diffraction and found to contain Amorphous Form.

### Example 5

### Purification of an Impure 9-Chloroacetamidominocycline

Some impure 9-chloroacetamidominocycline was mixed with water and pH of the mixture was adjusted at ˜5.3. The resulted solution was extracted several times with dichloromethane and the combined organic extracts were washed with water, dried over sodium sulfate and filtered to afford a solution of much purer 9-chloroacetamidominocycline. Eventually, this solution can be treated as described in Examples 3 or 4 in order to isolate the purified compound in the desired form.

### Example 6

### Preparation of Tigeycline

The product from Example 2 or 3 was mixed with an excess of t-butylamine, which serves also as a solvent, and 10% w/w of sodium iodide and the resulted mixture was stirred at ambient temperature overnight. Upon completion of the reaction the excessive amine was evaporated to dryness and the residue was covered with 100 ml of water. The resulted mixture was adjusted at pH 5 and extracted with dichloromethane several times to remove most of the impurities. The aqueous phase was then adjusted at pH ˜7.2 and extracted with dichloromethane several more times. The combined organic extracts of the second series were dried over sodium sulfate, filtered and evaporated to dryness. The residual orange powder was dried overnight at 40° C. under vacuum to afford pure Tigecycline in about 50% yield.

### Example 7

### Isolation of 9-Chlorocacetamidominominocycline an Acid Addition Salt and Conversion to Tigecycline

5 eq. of HCl in ether (based on the starting 9-aminominocycline) was added to the organic solution from Example 1 or 2. Precipitation started immediately and the suspension was stirred for an hour. The solid was collected by vacuum filtration and dried under vacuum at 40° C. overnight. 9-chloroacetamidominocycline hydrochloride thus obtained was characterized by high chromatographic purity but lower molar yield than in Examples 3 and 4.

The product was subjected to the same procedure as described in Example 6 but resulted in Tigecycline of slightly lower quality with respect to that of the product of Example 6. Additionally, the yield in this case was as low as ˜35%.

We claim:

1. A solid (4S,4aS,5aR,12aS)-9-haloacetamido-4,7-bis(dimethylmino)-1,4,4a,5,5a,6,11,12a-octahydro-3,10,12,12a-tetrahydroxy-1,11-dioxo-2-naphthacenecarboxamide (9-haloacetamidominocycline) in the form of a free acid or amine addition salt.

2. The solid 9-haloacetamidominocycline of claim 1, wherein the 9-haloacetamidominocyline is 9-chloracetamidominocycline or 9-bromoacetamidominocycline.

3. A 9-haloacetamidominocycline in the form of a free acid or amine addition salt having a purity of at least 90% by area as measured by HPLC.

4. The 9-haloacetamidominocyline of claim 3, wherein the purity is at least 95% by area as measured by HPLC.

5. The 9-haloacetamidominocyline of claim 4, wherein the purity is at least 98% by area as measured by HPLC.

6. The 9-haloacetamidominocycline of claim 3, wherein the has not more than about 10% of the corresponding C-4 epimer.

7. The 9-haloacetamidominocyline of claim 3, wherein the 9-haloacetamidominocyline is 9-chloracetamidominocycline or 9-bromoacetamidominocycline.

8. A process for preparing a 9-haloacetamidominocycline in the form of a free acid or amine addition salt comprising:
a) providing a solution comprising 9-haloacetamidominocycline and water;
b) adjusting the pH between about 4 to about 7; and
c) extracting 9-haloacetamidominocycline using a water immiscible organic solvent; and
d) optionally recovering solid 9-haloacetamidominocycline in free acid form.

9. The process of claim 8, wherein the 9-haloacetomidominocyline is 9-chloracetamidominocycline or 9-bromoacetamidominocycline.

10. The process of claim 8, wherein the solution in step a) further comprises a water miscible organic solvent.

11. The process of claim 10, wherein the water miscible organic solvent is DMA, DMI, DMF, NMP, DMPU or mixtures thereof.

12. The process of claim 8, wherein the pH is about 5 to about 6.

13. The process of claim 12, wherein the pH is about 5.0 to about 5.6.

14. The process of claim 8, wherein adjusting the pH comprises adding an inorganic or organic base or a basic aqueous solution to the mixture of step a).

15. The process of claim 14, wherein the pH is adjusted using ammonium hydroxide.

16. The process of claim 8, wherein the water immiscible organic solvent is selected from the group consisting of: a linear or branch-chain C₂₋₈ ether, a linear or branch-chain C₃₋₆ ketone, a linear or branch-chain C₅₋₁₂ ester, a halogenated hydrocarbon and mixtures thereof.

17. The process of claim 16, wherein the water immiscible organic solvent is selected from the group consisting of: iso-butyl acetate, methyl iso-butyl ketone, methyl t-butyl ether, dichloromethane and mixtures thereof.

18. The process of claim 17, wherein the water immiscible organic solvent is dichloromethane.

19. The process of claim 8, wherein the solid 9-haloacetamidominocycline is recovered in step d), recovering comprising exposure to a drying agent.

20. The process of claim 19, wherein the drying agent is sodium sulfate or magnesium sulfate.

21. The process of claim 8, wherein the solid 9-haloacetamidominocycline is recovered in step d), recovering comprising precipitation of the free acid.

22. The process of claim 21, wherein precipitation comprises admixing an antisolvent.

23. The process of claim 22, further comprising reducing the volume of the solution containing 9-haloacetamidominocycline.

24. The process as in claim 22, wherein the antisolvent is a C₅-C₈ saturated hydrocarbon.

25. The process of claim 24, wherein the C₅-C₈ saturated hydrocarbon is n-hexane or cyclohexane.

26. The process of claim 8, further comprising conversion to an acid or amine addition salt or adduct.

27. The process as in claim 26, wherein conversion to an amine addition salt or adduct comprises: admixing about 1 to about 20 molar equivalents of an amine; and
recovering 9-haloacetamidomidominocycline having a purity of at least about 90% by area as measured by HPLC in salt or adduct form.

28. The process as in claim 27, wherein the molar amount is about 1 to about 10

29. The process as in claim 28, wherein the molar amount is about 2 to about 5.

30. The process as in claim 27, wherein the amine is selected from the list consisting of: t-butylamine, triethylamine, isopropylamine.

31. The process of claim 26, further comprising converting 9-haloacetamidominocycline in the form of an acid or amine salt or adduct having a purity of at least about 90% by area as measured by HPLC to Tigecycline.

32. The process of claim 8, wherein extracting with a water immiscible organic solvent in step c) is repeated about 3 to about 5 times.

33. The process of claim 8, further comprising converting the 9-haloacetamidominocycline having a purity of at least about 90% by area as measured by HPLC to Tigecycline.

34. The process of claim 33, wherein the substantially pure 9-haloacetamidominocycline is converted to Tigecycline without isolating the substantially pure 9-haloacetamidominocycline in solid form, comprising reacting the 9-haloacetamidominocycline with t-butylamine.

35. The process of claim 8, wherein the solid 9-haloacetamidominocycline is recovered in step d), recovering of the 9-haloacetamidominocycline in a free acid form comprises:
a) providing an organic solution of 9-haloacetamidominocycline;
b) reducing the volume of the solution;
c) admixing at least a 3 fold volume of the solution in b) of an antisolvent to obtain a precipitate; and
d) optionally recovering the precipitated 9-haloacetamidominocycline.

36. The process in 35, wherein the antisolvent is a saturated C₅-C₈ hydrocarbon

37. The process of claim 36, wherein the C₅-C₈ saturated hydrocarbon is n-hexane or cyclohexane.

38. The process of claim 35, wherein the 9-haloacetamidominocycline is 9-chloroacetamidominocycline or 9-bromoacetamidominocycline.

39. The process of claim 35, wherein the 9-haloacetamidominocycline has a purity of at least about 90% by area as measured by HPLC.

40. The process of claim 35, further comprising converting the 9-haloacetamidominocycline having a purity of at least about 90% by area as measured by HPLC to Tigecycline

41. The process of claim 40, wherein the 9-haloacetamidominocycline is converted to Tigecycline without recovering the 9-haloacetamidominocycline in solid form, comprising reacting the 9-haloacetamidominocycline with t-butylamine.

42. A process for preparing 9-haloacetamidominocycline in a salt or adduct form comprising:
a) providing an organic solution of 9-haloacetamidominocycline having a purity of at least about 90% by area as measured by HPLC;
b) admixing about 1 to about 20 equivalents of an amine or acid; and
c) optionally recovering 9-haloacetamidominocycline in salt or adduct form having a purity of at least about 90% by area as measured by HPLC.

43. The process of claim 42, wherein the 9-haloacetamidominocycline is 9-chloroacetamidominocycline or 9-bromoacetamidominocycline.

44. The process of claim 42, wherein step b) comprises admixing an amine selected from t-butylamine, triethylamine, and isopropylamine.

45. The process of claim 42, wherein step b) comprises admixing an acid selected from hydrochloric acid, hydrobromic acid and trifluoroacetic acid.

46. The process of claim 42, further comprising converting the 9-haloacetamidominocycline having a purity of at least about 90% by area as measured by HPLC to Tigecycline.

47. The process of claim 46, wherein the 9-haloacetamidominocycline is converted to Tigecycline without recovering the 9-haloacetamidominocycline in solid form, comprising reacting the 9-haloacetamidominocycline with t-butylamine.

48. A process of preparing a glycylcycline from a 9-haloacetamidominocycline having a purity of at least about 90% by area as measured by HPLC.

49. The process of claim 48, wherein the 9-haloacetamidominocycline is 9-chloroacetamidominocycline or 9-bromoacetamidominocycline.

50. The process of claim 48, wherein the glycylcycline is Tigecycline.
